(19) United States
(12) Patent Application Publication
(10) Pub. No.: US 2023/0284885 A1
(43) Pub. Date: Sep. 14, 2023

(54) CONTROLLABLE BENDING TUBE AND ENDOSCOPE DEVICE THEREOF

(71) Applicant: ALTEK BIOTECHNOLOGY CORPORATION, Hsinchu City (TW)

(72) Inventor: Yu-Cheng Huang, Hsinchu City (TW)

(73) Assignee: ALTEK BIOTECHNOLOGY CORPORATION, Hsinchu City (TW)

(21) Appl. No.: 18/104,252

(22) Filed: Jan. 31, 2023

Related U.S. Application Data
(60) Provisional application No. 63/318,786, filed on Mar. 11, 2022.

(51) Int. Cl. A61B 1/005; A61B 1/00

(52) U.S. Cl. CPC A61B 1/0057; A61B 1/00096

(57) ABSTRACT

A controllable bending tube is applied to an endoscope device having an endoscope lens module disposed on an end of the controllable bending tube and includes a first wire, a second wire, and a plurality of helical coil portions connected in series to integrally form a helical tube body with a helical pitch. A first retaining slot and a second retaining slot are formed at first sides and second sides of at least two of the plurality of helical coil portions respectively, and two first nodes protrudes axially at third sides and fourth sides of the at least two of the plurality of helical coil portions respectively. The first and second wires are movably disposed through the first and second retaining slots for controlling a bending direction of the helical tube body.

### CROSS REFERENCE TO RELATED APPLICATIONS

This application claims the benefit of U.S. Provisional Application No. 63/318,786, filed on Mar. 11, 2022. The content of the application is incorporated herein by reference.

### BACKGROUND OF THE INVENTION

### 1. Field of the Invention

The present invention relates to a controllable bending tube and an endoscope device thereof, and more specifically, to a controllable bending tube having an integrated helical tube body and an endoscope device thereof.

### 2. Description of the Prior Art

An endoscope device is a long and flexible tube apparatus, and mainly includes an image capture device, a light source and a controllable bending tube. After the endoscope device is electrically connected to a monitor, organs in human's body can be imaging by the endoscope device and shown on the monitor, so that the endoscope device can be applied to medical inspection and treatment. In general, the endoscope device utilizes a control bar to be connected to two wires threaded through the controllable bending tube, such that rotation of the control bar can apply tension to one wire and release tension from another wire, thereby controlling a bending direction of the endoscope device (e.g., bending the endoscope device in an upward or downward direction) for the subsequent medical inspection.

However, it is difficult to thread the wires through the controllable bending tube smoothly and quickly since the wires and the controllable bending tube are both small-sized components, so as to cause a time-consuming and strenuous wire assembly process. Furthermore, the controllable bending tube of the endoscope device usually adopts a complicated detachable tube assembly design for achieving the bendable effect, so as to increase the manufacturing cost of the endoscope device and the risk of fracture of the controllable bending tube.

### SUMMARY OF THE INVENTION

The present invention provides a controllable bending tube applied to an endoscope device. An endoscope lens module of the endoscope device is disposed on an end of the controllable bending tube. The controllable bending tube includes a plurality of helical coil portions, a first wire, and a second wire. The plurality of helical coil portions is connected in series to integrally form a helical tube body with a helical slot formed thereon along an axial direction of the helical tube body. The helical slot has a helical pitch. Each helical coil portion has a first side and a second side opposite to each other and has a third side and a fourth side located between the first side and the second side and opposite to each other. A first retaining slot and a second retaining slot are formed at the first sides and the second sides of at least two of the plurality of helical coil portions respectively, and two first nodes protrude along the axial direction at the third sides and the fourth sides of the at least two of the plurality of helical coil portions respectively. The first wire is movably disposed through the first retaining slots. The second wire movably disposed through the second retaining slots for controlling a bending direction of the helical tube body cooperatively with the first wire.

The present invention further provides an endoscope device including the aforesaid controllable bending tube, a control bar, and an endoscope lens module. The control bar is connected to the first wire and the second wire for controlling the bending direction of the helical tube body cooperatively with the first wire and the second wire. The endoscope lens module is disposed on an end of the controllable bending tube.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a partial internal diagram of an endoscope device according to an embodiment of the present invention.

FIG. 2 is a partial cross-sectional diagram of the endoscope device in FIG. 1 along a cross-sectional line I-I.

FIG. 3 is a partial enlarged diagram of a controllable bending tube in FIG. 1 at another viewing angle.

FIG. 4 is a partial enlarged diagram of a controllable bending tube according to another embodiment of the present invention.

FIG. 5 is a partial side view of an endoscope device according to another embodiment of the present invention.

FIG. 6 is a partial enlarged diagram of a controllable bending tube according to another embodiment of the present invention.

### DETAILED DESCRIPTION

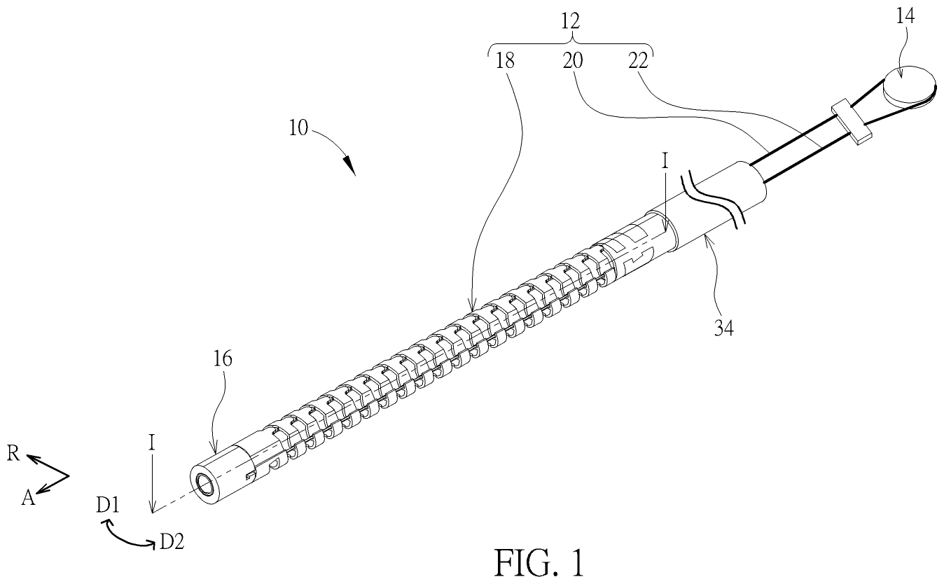

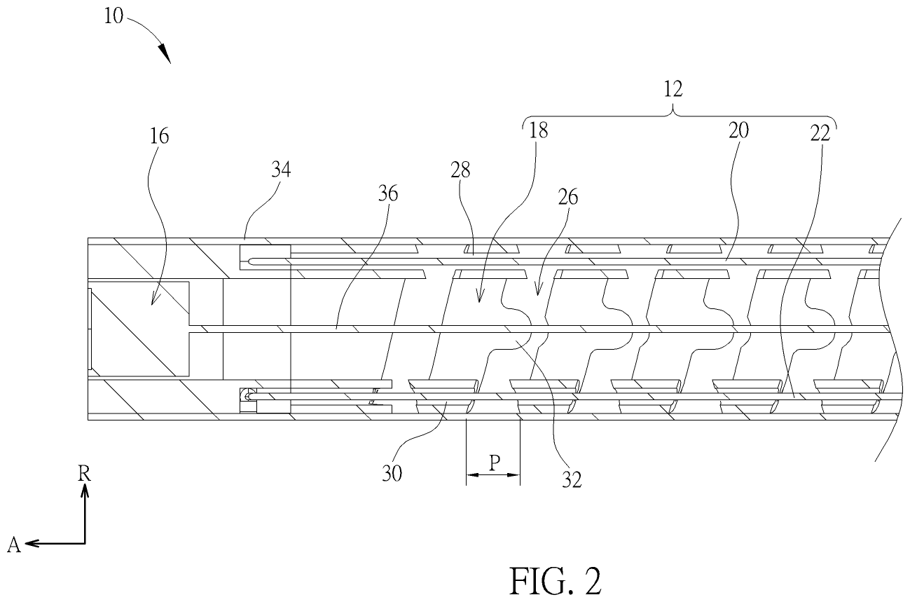

Please refer to FIG. 1 and FIG. 2. FIG. 1 is a partial internal diagram of an endoscope device 10 according to an embodiment of the present invention. FIG. 2 is a partial cross-sectional diagram of the endoscope device 10 in FIG. 1 along a cross-sectional line I-I. As shown in FIG. 1 and FIG. 2, the endoscope device 10 includes a controllable bending tube 12, a control bar 14, and an endoscope lens module 16. The controllable bending tube 12 includes a plurality of helical coil portions 18, a first wire 20, and a second wire 22. The plurality of helical coil portions 18 is connected in series to integrally form a helical tube body 24 with a helical slot 26 formed thereon along an axial direction A of the helical tube body 24, and the helical slot 26 has a helical pitch P, wherein an extending direction of the helical slot 26 is not perpendicular to an axial direction A of the helical tube body 24 (as shown in FIG. 2). In this embodiment, the helical tube body 24 could be preferably made of metal material (e.g., stainless steel, copper, aluminum or memory alloy) and formed by a laser cutting process, but not limited thereto. That is to say, the helical tube body 24 could be also formed by other tube forming process. For example, the helical tube body 24 could be made of plastic material (e.g., PP (Polypropylene) or POM (Polyoxymethylene)) and formed by a plastic mold injection process.

The endoscope lens module 16 is disposed on an end of the controllable bending tube 12, and the control bar 14 (preferably a rotating cam mechanism as shown in FIG. 1, but not limited thereto) is connected to the first wire 20 and the second wire 22. The first wire 20 and the second wire 22 are movably disposed through two sides of the helical tube body 24 and preferably fixed to the end of the controllable bending tube 12. In such a manner, rotation of the control bar 14 can apply tension to one wire and release tension from another wire, thereby controlling a bending direction of the endoscope device 10 to operate the endoscope lens module 16 to perform endoscope imaging (e.g., imaging organs in human's body) for the subsequent medical inspection. For example, clockwise rotation of the control bar 14 in FIG. 1 can apply tension to the first wire 20 and release tension from the second wire 22 for bending the endoscope device 10 in a rightward direction D1 relative to the axial direction A, and counterclockwise rotation of the control bar 14 in FIG. 1 can apply tension to the second wire 22 and release tension from the first wire 20 for bending the endoscope device 10 in a leftward direction D2 relative to the axial direction A. As for the wire controlling design of the control bar 14 and the imaging design of the endoscope lens module 16, the related description is commonly seen in the prior art and omitted herein. To be noted, the wire fixing design of the present invention is not limited to the aforesaid embodiment. For example, in another embodiment, the present invention could adopt the design that the first wire 20 and the second wire 22 are fixed to the endoscope lens module 16 instead of being fixed to the end of the controllable bending tube 12. As for which design is adopted, it depends on the practical application of the present invention.

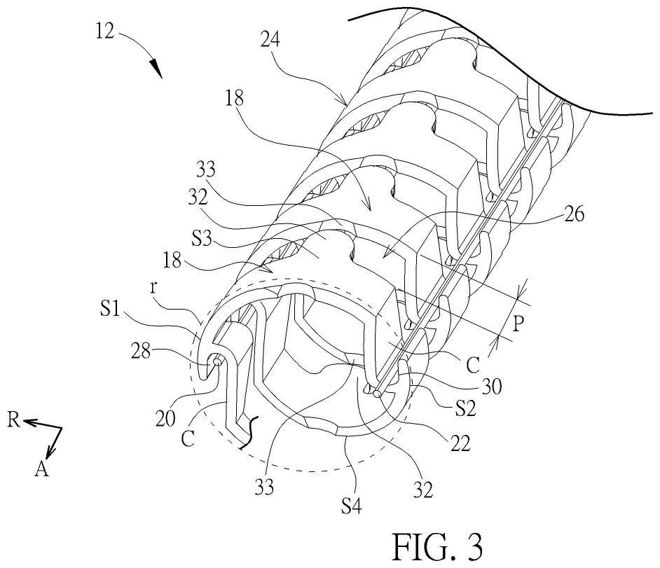

More detailed description for the helical structural design of the controllable bending tube 12 is provided as follows. Please refer to FIG. 2 and FIG. 3. FIG. 3 is a partial enlarged diagram of the controllable bending tube 12 in FIG. 1 at another viewing angle. As shown in FIG. 2 and FIG. 3, each helical coil portion 18 has a first side S1 and a second side S2 opposite to each other and has a third side S3 and a fourth side S4 located between the first side S1 and the second side S2 and opposite to each other, wherein a central angle corresponding to the helical coil portion 18 relative to the axial direction A of the helical coil body 24 could be preferably equal to 360° (as shown in FIG. 3, but not limited thereto). A first retaining slot 28 and a second retaining slot 30 are formed at the first sides S1 and the second sides S2 of at least two of the plurality of helical coil portions 18 respectively (in this embodiment, each helical coil portion 18 could have the first retaining slot 28 and the second retaining slot 30 respectively formed at the first side S1 and the second side S2, but the present invention is not limited thereto), and two first nodes 32 protrude along the axial direction A at the third sides S3 and the fourth sides S4 of the at least two of the plurality of helical coil portions 18 respectively (in this embodiment, each helical coil portion 18 could have the two first nodes 32 respectively formed at the third side S3 and the fourth side S4, but the present invention is not limited thereto). As such, the first wire 20 and the second wire 22 can be movably disposed through the first retaining slots 28 and the second retaining slots 30 respectively, so as to complete the wire assembly process of the endoscope device 10 smoothly and quickly.

To be more specific, in this embodiment, the first retaining slot 28 and the second retaining slot 30 could be preferably formed within a circumferential range r (indicated by a dotted line in FIG. 3) of the helical tube body 24, for preventing protrusion of the first retaining slot 28 and the second retaining slot 30 outside the helical tube body 24 and reducing the overall tube volume of the helical tube body 24. Furthermore, the first retaining slot 28 and the second retaining slot 30 could respectively have a cutting plane C perpendicular to a radial direction R of the helical tube body 24, so as to guide the first wire 20 and the second wire 22 to be assembled in the first retaining slot 28 and the second retaining slot 30 more smoothly.

Moreover, the present invention could further adopt the slot constraining design for preventing the first wire 20 and the second wire 22 from sliding out of the first retaining slot 28 and the second retaining slot 30 accidentally when the control bar 14 rotates to move the first wire 20 and the second wire 22. For example, in this embodiment, an outlet direction of the first retaining slot 28 (downward as shown in FIG. 3) could be preferably perpendicular to the radial direction R of the helical tube body 24, and the outlet direction of the first retaining slot 28 could be preferably opposite to an outlet direction of the second retaining slot 30 (upward as shown in FIG. 3). On the other hand, in another embodiment, an outlet direction of at least one of the first retaining slots 28 and an outlet direction of at least one adjacent first retaining slot 28 could be opposite to each other for constraining the first wire 20 in the first retaining slots 28 steadily. As for which slot constraining design is utilized, it depends on the practical application of the endoscope device 10.

In addition, as shown in FIG. 1 and FIG. 3, the two first nodes 32 of the helical coil portion 18 could protrude toward the control bar 14 along the axial direction A of the helical tube body 24. As such, when rotation of the control bar 14 causes bending of the controllable bending tube 12 via the first wire 20 and the second wire 22, the pitch P can be reduced accordingly to make the two first nodes 32 detachably abut against an adjacent helical coil portion 18 for providing support points and bending fulcrums between the two adjacent helical coil portions 18, so as to efficiently reduce the risk of fracture of the helical tube body 24. Furthermore, in this embodiment, the adjacent helical coil portion 18 could have two recesses 33 formed thereon to be detachably matched with the two first nodes 32 for providing a preferable support. To be noted, as shown in FIG. 3, the first node 32 at the third side S3 is misaligned with the first node 32 at the fourth side S4 in the axial direction A of the helical tube body 24 for avoiding the stress concentration problem, so as to further reduce the risk of fracture of the helical tube body 24.

In practical application, as shown in FIG. 1 and FIG. 2, the endoscope device 10 could further include a film sleeve 34 (partially shown in FIG. 1 for exposing the controllable bending tube 12), and the film sleeve 34 is sleeved on the endoscope lens module 16 and the controllable bending tube 12 for external protection. Furthermore, the endoscope device 10 could further include a signal transmission element 36, and the signal transmission element 36 is disposed through the controllable bending tube 12 to be electrically connected to the endoscope lens module 16 for signal transmission. As for the signal transmission and circuit designs of the endoscope device 10, the related description is commonly seen in the prior art and omitted herein.

In summary, instead of the complicated detachable tube assembly design in the prior art, the present invention adopts the integral helical tube forming design to simplify the tube design of the endoscope device 10 and improve the structural elasticity of the controllable bending tube 12, so as to greatly reduce the manufacturing cost of the endoscope device 10 and the risk of fracture of the controllable bending tube 12. Furthermore, compared with the prior art adopting the wire threading design, the present invention adopts the outer retaining slot design to allow that the first wire 20 and the second wire 22 can be assembled in the first retaining slots 28 and the second retaining slots 30 at the two sides of the helical tube body 24 smoothly and quickly. In such a manner, the present invention can efficiently solve the prior art problem that the wire threading design causes a time-consuming and strenuous wire assembly process, so as to improve the wire assembly convenience and reduce the wire assembly time.

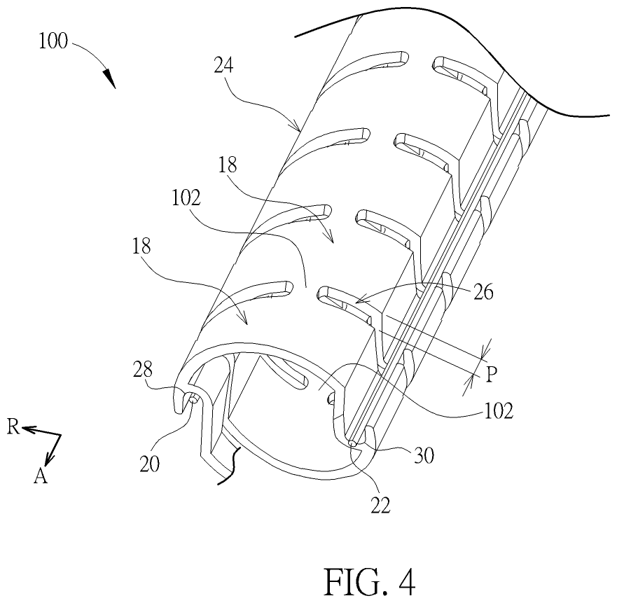

It should be mentioned that the present invention could also adopt the node connection design. For example, please refer to FIG. 4, which is a partial enlarged diagram of a controllable bending tube 100 according to another embodiment of the present invention. Components both mentioned in this embodiment and the aforesaid embodiments represent components with similar structures or functions, and the related description is omitted herein. As shown in FIG. 4, the controllable bending tube 100 includes the plurality of helical coil portions 18, the first wire 20, and the second wire 22, and each helical coil portions 18 is connected in series to integrally form the helical tube body 24 and has two first nodes 102 protruding along the axial direction A of the helical tube body 24 to be connected to an adjacent helical coil portion 18 for further improving the structural strength of the controllable bending tube 100. As for the other detailed description for the controllable bending tube 100 (e.g., the wire fixing design, the outer retaining slot design and the slot constraining design), it could be reasoned by analogy according to the aforesaid embodiment as shown in FIG. 3 and omitted herein.

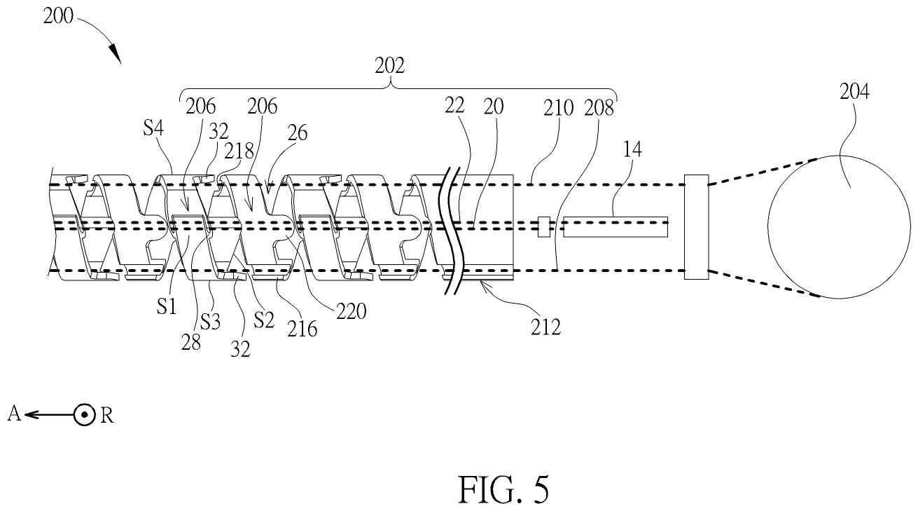

Furthermore, the present invention could also adopt the four wire controlling design. For example, please refer to FIG. 5, which is a partial side view of an endoscope device 200 according to another embodiment of the present invention. Components both mentioned in this embodiment and the aforesaid embodiments represent components with similar structures or functions, and the related description is omitted herein. For clearly showing the structural design of a controllable bending tube 202, the endoscope lens module 16, the film sleeve 34, and the signal transmission element 36 are omitted in FIG. 5, and the first wire 20, the second wire 22, a third wire 208, and a fourth wire 210 are depicted by dotted lines in FIG. 5.

As shown in FIG. 5, the endoscope device 200 could include the controllable bending tube 202, at least one control bar (two control bars 14, 204 shown in FIG. 5, but not limited thereto), the endoscope lens module 16, the film sleeve 34 and the signal transmission element 36. The controllable bending tube 202 includes a plurality of helical coil portions 206, the first wire 20, the second wire 22, the third wire 208 and the fourth wire 210, and the plurality of helical coil portions 206 is connected in series to integrally form a helical tube body 212 with the helical slot 26 formed thereon along the axial direction A.

In this embodiment, one of any two adjacent helical coil portions 206 has the first retaining slot 28 and the second retaining slot 30 (not shown in FIG. 5) formed at the first side S1 and the second side S2 respectively and has the two first nodes 32 protruding (preferably protruding toward the control bar 14, but not limited thereto) along the axial direction A at the third side S3 and the fourth side S4 respectively. Another of any two adjacent helical coil portions 206 has a third retaining slot 216 and a fourth retaining slot 218 formed at the third side S3 and the fourth side S4 respectively and has two second nodes 220 protruding (preferably protruding toward the control bar 14, but not limited thereto) along the axial direction A at the first side S1 and the second side S2 respectively. The first wire 20 and the second wire 22 are movably disposed through the first retaining slot 28 and the second retaining slot 30 respectively, and the control bar 14 is connected to the first wire 20 and the second wire 22. The third wire 208 and the fourth wire 210 are movably disposed through the third retaining slot 216 and the fourth retaining slot 218 respectively, and the control bar 204 is connected to the third wire 208 and the fourth wire 210.

In such a manner, as shown in FIG. 5, rotation of the control bar 14 can move the first wire 20 and the second wire 22 to bend the endoscope device 200 in the rightward or leftward direction relative to the axial direction A. During rotation of the control bar 14, the two first nodes 32 detachably abut against an adjacent helical coil portion 206 to provide support points and bending fulcrums, so as to reduce the risk of fracture of the helical tube body 212. Similarly, as shown in FIG. 5, rotation of the control bar 204 can move the third wire 208 and the fourth wire 210 to bend the endoscope device 200 in the upward or downward direction relative to the axial direction A. During rotation of the control bar 204, the two second nodes 220 detachably abut against another adjacent helical coil portion 206 to provide support points and bending fulcrums, so as to reduce the risk of fracture of the helical tube body 212. In summary, via the aforesaid four wire controlling design as shown in FIG. 5, the endoscope device 200 can achieve the four-way bending effect via rotation of the control bar 14 and the control bar 204, so as to improve the operational flexibility of the endoscope device 200. As for the other detailed description for the endoscope device 200 (e.g., the wire fixing design, the tube forming process, the outer retaining slot design, the slot constraining design, and the recess matching design), it could be reasoned by analogy according to the aforesaid embodiment as shown in FIG. 3 and omitted herein.

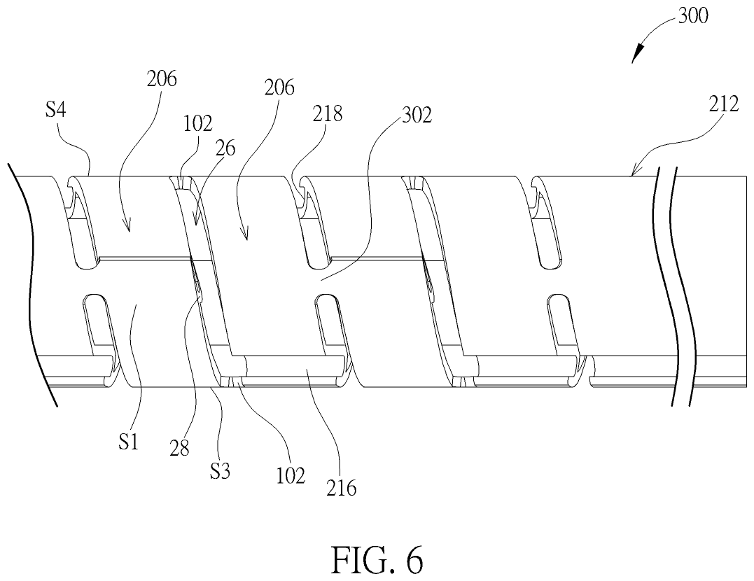

To be noted, the node connection design could be also applied to the aforesaid embodiment. For example, please refer to FIG. 6, which is a partial enlarged diagram of a controllable bending tube 300 according to another embodiment of the present invention. Components both mentioned in this embodiment and the aforesaid embodiments represent components with similar structures or functions, and the related description is omitted herein, and the first wire 20, the second wire 22, the third wire 208, and the fourth wire 210 are omitted in FIG. 6 for clearly showing the tube design of the controllable bending tube 300. As shown in FIG. 6, the controllable bending tube 300 includes the plurality of helical coil portions 206, the first wire 20, the second wire 22, the third wire 208, and the fourth wire 210, and the plurality of helical coil portions 206 is connected in series to integrally form the helical tube body 212. In this embodiment, as shown in FIG. 5, the helical coil portions 206 could have the two first nodes 102 formed at the third side S3 and the fourth side S4 respectively to be connected to an adjacent helical coil portions 206, and the adjacent helical coil portions 206 could have two second nodes 302 formed at the first side S1 and the second side S2 respectively (the second node 302 at the second side S2 not shown in FIG. 6) to be connected to another adjacent helical coil portion 206 for further improving the structural strength of the controllable bending tube 300. As for the other detailed description for the controllable bending tube 300 (e.g., the wire fixing design, the outer retaining slot design and the slot constraining design), it could be reasoned by analogy according to the aforesaid embodiment as shown in FIG. 3 and omitted herein.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A controllable bending tube applied to an endoscope device, an endoscope lens module of the endoscope device being disposed on an end of the controllable bending tube, the controllable bending tube comprising:
a plurality of helical coil portions connected in series to integrally form a helical tube body with a helical slot formed thereon along an axial direction of the helical tube body, the helical slot having a helical pitch, each helical coil portion having a first side and a second side opposite to each other and having a third side and a fourth side located between the first side and the second side and opposite to each other, a first retaining slot and a second retaining slot being formed at the first sides and the second sides of at least two of the plurality of helical coil portions respectively, and two first nodes protruding along the axial direction at the third sides and the fourth sides of the at least two of the plurality of helical coil portions respectively;
a first wire movably disposed through the first retaining slots; and
a second wire movably disposed through the second retaining slots for controlling a bending direction of the helical tube body cooperatively with the first wire.

2. The controllable bending tube of claim 1, wherein an outlet direction of at least one of the first retaining slots and an outlet direction of at least one adjacent first retaining slot are opposite to each other.

3. The controllable bending tube of claim 1, wherein the first retaining slot and the second retaining slot are formed within a circumferential range of the helical tube body, and the first retaining slot and the second retaining slot respectively have a cutting plane perpendicular to a radial direction of the helical tube body.

4. The controllable bending tube of claim 1, wherein an outlet direction of the first retaining slot is perpendicular to a radial direction of the helical tube body.

5. The controllable bending tube of claim 1, wherein the two first nodes protrude to detachably abut against an adjacent helical coil portion.

6. The controllable bending tube of claim 5, wherein the adjacent helical coil portion has two recesses formed thereon to be detachably matched with the two first nodes.

7. The controllable bending tube of claim 1, wherein the two first nodes protrude to be connected to an adjacent helical coil portion.

8. The controllable bending tube of claim 1, wherein the first node at the third side is misaligned with the first node at the fourth side in the axial direction of the helical tube body.

9. The controllable bending tube of claim 1, wherein the helical tube body is made of metal material and formed by a laser cutting process, or the helical tube body is formed by a plastic mold injection process.

10. The controllable bending tube of claim 1, wherein one of any two adjacent helical coil portions has the first retaining slot and the second retaining slot formed at the first side and the second side respectively and has the two first nodes protruding along the axial direction at the third side and the fourth side respectively, and another of any two adjacent helical coil portions has a third retaining slot and a fourth retaining slot formed at the third side and the fourth side respectively and has two second nodes protruding along the axial direction at the first side and the second side respectively; the controllable bending tube further comprises:
a third wire movably disposed through the third retaining slots; and
a fourth wire movably disposed through the fourth retaining slots for controlling a bending direction of the helical tube body cooperatively with the first wire, the second wire and the third wire.

11. The controllable bending tube of claim 10, wherein the first nodes detachably abut against the another of any two adjacent helical coil portions.

12. The controllable bending tube of claim 10, wherein the first nodes are connected to the another of any two adjacent helical coil portions.

13. The controllable bending tube of claim 10, wherein the first node at the third side is misaligned with the first node at the fourth side in the axial direction.

14. The controllable bending tube of claim 10, wherein the helical tube body is made of metal material and formed by a laser cutting process, or the helical tube body is formed by a plastic mold injection process.

15. An endoscope device comprising:
the controllable bending tube of claim 1;
a control bar connected to the first wire and the second wire for controlling the bending direction of the helical tube body cooperatively with the first wire and the second wire; and
an endoscope lens module disposed on an end of the controllable bending tube.

16. The endoscope device of claim 15, wherein the endoscope device further comprises a film sleeve, and the film sleeve is sleeved on the endoscope lens module and the controllable bending tube.
